(19) United States
(12) Patent Application Publication
(10) Pub. No.: US 2002/0177870 A1
(43) Pub. Date: Nov. 28, 2002

(54) Single lumen balloon catheter

(76) Inventors: Ivan Sepetka, Los Altos, CA (US); Maureen Bensing, Sunnyvale, CA (US); Diana Sanchez, Santa Clara, CA (US); Richard A. Helkowski, Redwood City, CA (US); Christopher Ken, San Mateo, CA (US); Mark Deem, Mountain View, CA (US); Maria ` Aboytes, East Palo Alto, CA (US); Hanson S. Gifford III, Woodside, CA (US)

Correspondence Address: MORRISON & FOERSTER LLP, 755 PAGE MILL RD, PALO ALTO, CA, 94304-1018, US

(21) Appl. No.: 09/865,998

(22) Filed: May 25, 2001

(51) Int. Cl. A61M 29/00

(52) U.S. Cl. 606/194; 604/99.04

(57) ABSTRACT

This invention is a surgical device. In particular, it is a low profile, single lumen catheter preferably having a movable seal or seat that allows the balloon to be inflated by sealing against the movable guidewire or against itself. An additional variation of the invention includes a non-removable guidewire situated in the catheter body in such a way to provide or add stiffness to the otherwise flexible distal section of the catheter during a procedure. An enhanced strain relief transition joint between significantly stiffer proximal section and the more flexible distal section is provided. Finally, methods of using the inventive balloon catheter are also shown.

### FIELD OF THE INVENTION

This invention is a surgical device. In particular, it is a low profile, single lumen catheter preferably having a movable seal or seat that allows the balloon to be inflated by sealing against the movable guidewire or against itself. An additional variation of the invention includes a non-removable guidewire situated in the catheter body in such a way to provide or add stiffness to the otherwise flexible distal section of the catheter during a procedure. An enhanced strain relief transition joint between the significantly stiffer proximal section and the more flexible distal section is provided. The catheter may be used in any service, but it is especially useful when sized and selected as a microcatheter in neurovascular procedures. Finally, methods of using the inventive balloon catheter are also shown.

### BACKGROUND OF THE INVENTION

This invention relates generally to medical balloon catheters, their structures, and methods of using them. In particular, the present invention relates to the construction of both large and small diameter, typically braid-reinforced balloon catheters having controlled flexibility, a soft distal tip and a typically elastomeric balloon near the distal tip for the partial or total occlusion of a vessel. This catheter uses a movable seal to direct fluid to or to bleed fluid from the balloon. The inventive catheter may be used for a wide variety of medical applications, such as interventional cardiological, peripheral, or neuroradiology procedures, but are particularly useful in intercranial selective catheterization.

Medical catheters are used for a variety of purposes, including interventional therapy, drug delivery, diagnosis, perfusion, and the like. Catheters for each of these purposes may be introduced to the target sites within a patient's body by guiding the catheter through the vascular system, and a wide variety of specific catheter designs have been proposed for different uses.

Of particular interest to the present invention are large lumen balloon catheters used in supporting procedures, in turn, using small diameter tubular access catheters. Such procedures include diagnostic and interventional neurological techniques, such as the imaging and treatment of aneurysms, tumors, arteriovenous malformations, fistulas, and the like. Practical treatment of embolic stroke is novel. The neurological vasculature places a number of requirements on the small catheters that may be used. The catheters should be quite small. The blood vessels in the brain are frequently as small as several millimeters, or less, requiring that the intervening catheters have an outside diameter as small as one French (0.33 millimeters). In addition to small size, the brain vasculature is highly tortuous, requiring that neurological catheters be very flexible, particularly at their distal ends, to pass through the regions of tortuosity. The blood vessels of the brain are quite fragile, so it is desirable that the catheter have a soft, non-traumatic exterior to prevent injury. The advent of interventional radiology and its sub-practice, interventional neuroradiology, as a viable treatment alternatives in various regions of the body having tortuous vasculature often surrounded by soft organs, has produced demands on catheterization equipment not placed on devices used in PCTA and PTA. The need for significantly smaller diameter devices and particularly those which have variable flexibility and are able to resist kinking is significant.

Typical of the single lumen balloon catheter devices found in the literature are U.S. Pat. No. 5,776,099, to Tremulis; U.S. Pat. No. 6,074, 407, to Levine et al; U.S. Pat. Nos. 6,096,055, 5,683,410, and U.S. Pat. No. 5,304,198, all to Samson; U.S. Pat. No. 6,017,323, to Chee; U.S. Pat. No. 6,193,686, to Estrada et al; U.S. Pat. No. 6,090,126, to Burns; and U.S. Pat. No. 5,364,354, to Walker et al. None of the cited art suggests the structure found in the inventive catheter.

### SUMMARY OF THE INVENTION

This invention has several variations. It desirably is a low profile balloon catheter for use with a removable guide wire and is made up of a catheter body having a distal end, a proximal end, and a passageway for inflation of a balloon at the distal end of the inflation lumen. The balloon is located near said distal end and is filled with a fluid when a movable seal cooperatives to seal the inflation passageway and, of course, fluid is introduced into the passageway. The balloon may be compliant.

There are several variations of the seal. One seal is inflatable employs a fluid supply lumen independent of the balloon inflation passageway. It may be situated within the passageway and upon inflation of the seal closes against the removable guide wire.

Another variation is both self-closing and sealable against the removable guide wire when that guide wire passes through said movable seal. That variation of the seal may be distal of the balloon and, indeed, may be an extension, perhaps an everted extension, of the balloon.

The seal assemblage of this invention may include an auxiliary seal for initially sealing the passageway against the guide wire while the balloon itself uses its own distal end to form the seal. The distal end closes both against the guide wire and is self-closing against itself upon introduction of a fluid into the balloon.

In another variation, the seal is closed and permits inflation of the balloon when fluid is introduced into the fluid passageway. When the seal is penetrated by the guide wire, the inflation passes through the seal and balloon deflates. The seal may have a mating surface that is adapted to cooperate with the guide wire to provide openings in the seal adjacent to the guide wire. That mating surface may be sinusoidal or other suitable shape.

The balloon catheter has a catheter shaft and the proximal section of that shaft typically is different in many aspects from the distal section. For instance, the proximal section may have a diameter larger than that of the distal section or may have a different flexibility.

The balloon catheter may be used for a variety of purposes. The catheter may be of a flexibility, length, and diameter appropriate for a neurovascular microcatheter or for a guide catheter or other selected balloon catheter style. Another significant variation of the inventive low profile balloon catheter involves a generally non-removable guide wire and a seal to fill the balloon. The guide wire may be adapted to provide selectable axial stiffness to the flexible distal section of the catheter. The stiffness of the guide wire may be continuously and incrementally variable. The guide wire may have a variable diameter. The proximal portion of said catheter may be a hypotube.

Another aspect of the invention involves a strain relief joint between a first comparatively stiff section adjacent a second comparatively flexible section, perhaps both tubing members. The first stiff section may have a diameter different than that the second flexible section. The joint includes a corkscrew-shaped component wound over said joint that adheres to both the first and to said second sections. The first and second sections may be polymeric. Preferred are joints at least partially wrapped by a metallic ribbon of stainless steel or a superelastic alloy. It is desirable to adhere the the metallic ribbon and the corkscrew-shaped component to the tubing sections via a first molten and then-solidified polymer. One or more shrink-wrapped polymeric coverings over the joints are desired.

### BRIEF DESCRIPTION OF THE DRAWINGS

FIG. 1 shows a view of the inventive catheter.

FIG. 2 shows partial cross-section of a variation of the inventive balloon catheter.

FIG. 3 shows in partial cross-section, details of the distal tip of the inventive balloon catheter.

FIG. 4A shows, in partial cross-section, details of the distal tip of the inventive balloon catheter having an inflatable seal.

FIG. 4B shows, in cross section, the variation shown in FIG. 4A.

FIG. 4C shows a partial longitudinal cross section of the variation of the catheter shown in FIGS. 4A and 4B with the seal inflated.

FIGS. 5A, 5B, and 5C show a variation of the inventive catheter in which the balloon is variously sealing against the introduced guidewire and against itself. FIG. 5A shows the balloon in a deflated condition. FIG. 5B shows the balloon in an inflated condition with the seal seated against the guidewire. FIG. 5C shows the balloon inflated against itself rather than against the guidewire.

FIGS. 6A, 6B, 6C, and 6D show variations of the balloon catheter having a seal distal on the balloon and formed of material extending from the balloon. FIG. 6A shows the instance in which the balloon is not inflated. FIG. 6B shows the seal closed against the guidewire with the balloon inflated. FIG. 6C shows the balloon inflated with the seal self-closing. FIG. 6D shows a variation of the seal having a single layer of seal material in contrast to the everted, multilayer design of FIGS. 6A, 6B, and 6C.

FIGS. 7A-7F show a version of the balloon catheter having a self-closing distal seal which is only opened by introduction of a guidewire through the seal. FIG. 7A shows a deflated balloon prior to the introduction of inflation fluid. FIG. 7B shows an end view of the FIG. 7A variation. FIG. 7C shows a deflated balloon with a guidewire penetrating the distal seal. FIG. 7D shows an end view of that instance. FIG. 7E shows the balloon inflated and the end seal closed. FIG. 7F shows an end view of the instance shown in FIG. 7E.

FIG. 8 shows a variation of the inventive balloon catheter having a captive guidewire.

FIG. 9 shows another variation of that shown in FIG. 8.

### DESCRIPTION OF THE INVENTION

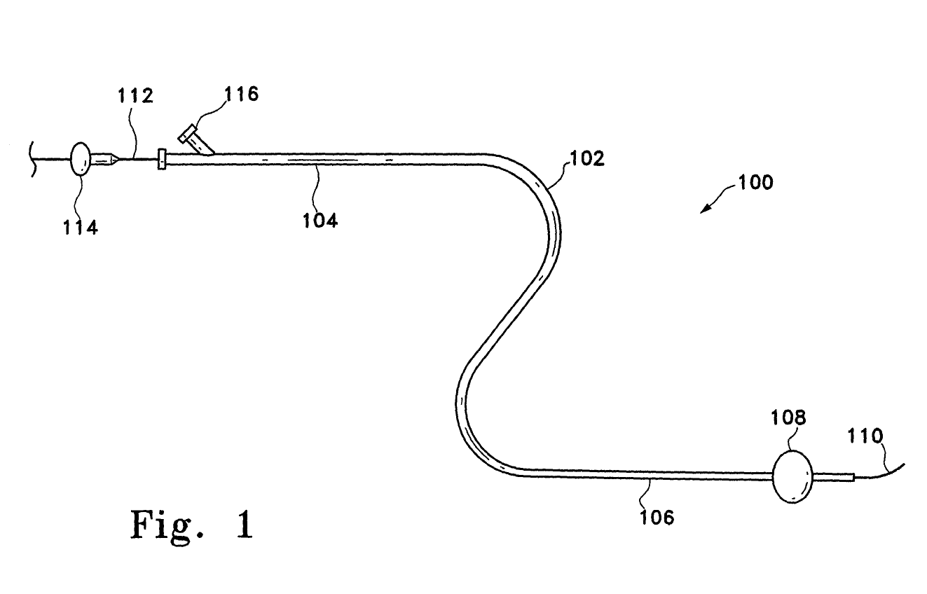

FIG. 1 shows a generic layout of the inventive catheter (100). Specifically, catheter (100) has a catheter body (102) which has one or more catheter sections typically having different flexibility. The proximal portion (104) of the catheter body is desirably quite stiff and the more distal portion (106) of the catheter body is, by comparison, significantly more flexible. The inflatable membrane or balloon (108) is quite distal on the catheter (100). Guidewire (110) having a distal tip is shown in the Figure. The guidewire (110) may be removable and is adapted to cooperate with seals found interior to the lumen catheter body (102) to inflate the balloon (108). At the proximal end of the balloon catheter may be found the proximal end (112) of the guidewire (110) and a torquer (114) for torquing or twisting the guidewire for its movement through the vasculature.

Typical fluid connections are also used. For instance, a fluid connector (116), e.g., a “Luer-Lok”, for introduction of the balloon inflation fluid is also shown.

The overall length of the inventive catheter (100) preferably is in the range of 100 to 225 cm, preferably 175 to 210 cm. Since the preferable use of this inventive balloon catheter is in the neurovasculature, the diameter of the catheter body distally is 25 to 40 mils, preferably 30 to 35 mils. Where the catheter body is stepped, the diameter of the more proximal section preferably is 40 mils to 55 mils, most preferably 45 to 50 mils in diameter. The axial length of the balloon (108) desirably is 10 to 25 mm, more 10 to 20 mm, and most preferably about 15 mm in length. The length of the more flexible distal section (106) is preferably from 15 to 40 cm in length, more preferably 15 to 25 cm, and most preferably about 20 cm in length. The more proximal section may be made up of one or more subsections of varying construction, and perhaps differing stiffness, but in any event makes up the rest of the overall catheter length.

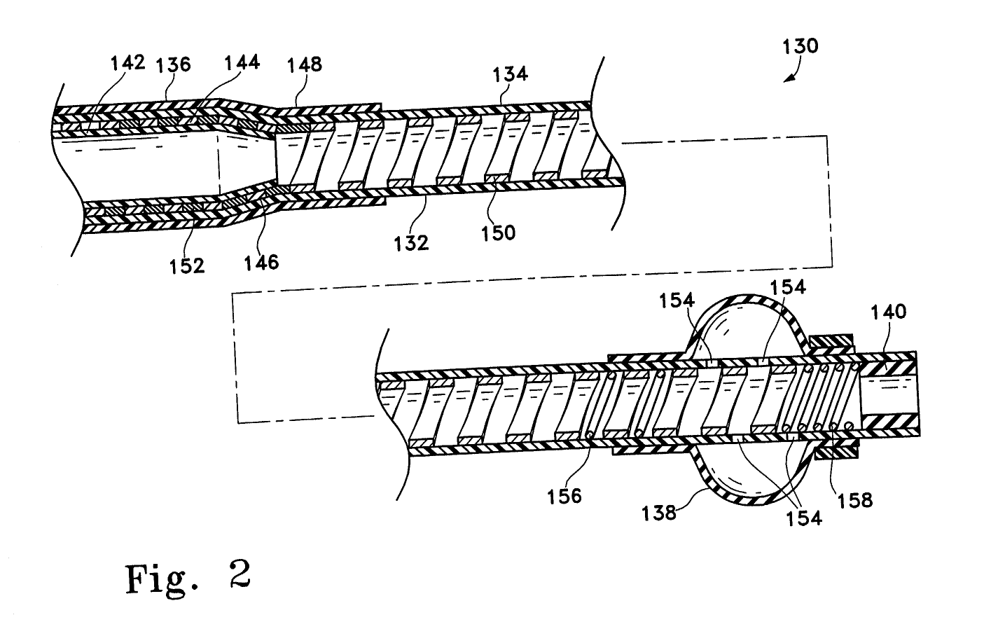

FIG. 2 shows in partial cross-section of the inventive catheter (130) showing a highly desirable construction of catheter body (132) with more flexible distal section (134) and a stiffer proximal section (136). A balloon (138) in inflated condition is also shown, as is seal (140). A movable guidewire, that is necessary for inflation of the balloon as shown, has been removed for a more thorough explanation of the construction of the catheter body (130).

In this variation, the most proximal portion of the proximal section involves a quite stiff inner layer (142) quite desirably of a material such as polyaryletheretherketone (PEEK) and variations of such ketone-based resins such as PEKK, PEKEKK, and the like. Polysulphones, including polyethersulphones, and polyphenylsulphones and various members of the Nylon family may be used. A metallic tube such as a hypotube is also suitable. Just distal of proximal inner liner (142) is an inner liner (144), preferably of material which is intermediate in flexibility between the inner liner (142) and distal section (134). Typical of such a material would be high density polyethylene (HDPE). Thermoplastics such as HDPE are desirable in that junction between larger proximal section and the smaller diameter distal section (136) may be easily fabricated.

In this desired variation, substantially all or a significant portion of the catheter assembly (130) is covered by an irradiated shrink-wrap layer (146) of shrink-wrap of polyolefin or other similar material such as low density polyethylene (LDPE). An auxiliary covering of another shrink-wrap of polyolefin (148) (such as LDPE or LLDPE) may also be seen in the FIG. 2 depiction. The auxiliary or outer covering (148) is placed over a majority of or all of the proximal section of catheter assembly (130) to provide additional stiffness to the proximal portion and to provide stability and some initial measure strain resistance to the junction between proximal portion (136) and distal portion (132).

Further, this variation of the inventive catheter utilizes an anti-kinking member (150). The variation shown here includes a ribbon coil which is desirably continuous for a significant length of the catheter, desirably for the total length. Any of the ribbon and wire discussed here may be variously metallic (e.g., stainless steels or superelastic alloys such as nitinol) or polymeric. The polymers may be single phase, e.g., such as monofilament line, or multiple strands bundled or woven together. These components may be made of a mixture of materials, e.g., superelastic alloy and stainless steel components or of LCPs. Preferred because of cost, strength, and ready availability are the stainless steels (SS304, SS306, SS308, SS316, SS318, etc.) and tungsten alloys. Especially preferred is stainless steel and, in particular, SS304-V. In certain applications, particularly in smaller diameter devices, more malleable metals and alloys, e.g., gold, platinum, palladium, rhodium, etc. may occasionally be, but then in combination with other materials for strength. A platinum alloy with a few percent of tungsten is sometimes used because of its high radio-opacity.

When using a super-elastic alloy in any of the component tubing members, an additional step may be desirable to preserve the shape of the stiffening braid or coil. For instance, after a structure such as a coil has been wound or a braid has been woven, some heat treatment may be desirable. Braids and coils that are not treated this way may unravel during subsequent handling or may undertake changes in diameter or spacing during that handling. In any event, the braids or coils are placed on a heat-resistant mandrel and placed in an oven at a temperature of, e.g., 650° to 750° F. for a few minutes. This treatment may anneal the material in the constituent ribbon or wire but in any event provides it with a predictable shape for subsequent assembly steps. After heat-treatment, the braid or braid retains its shape and most importantly the alloy should retain its super-elastic properties.

The antikinking member (150) preferably is formed from ribbons of stainless steel, superelastic alloys such as nitinol, or polymeric constructs. Although the braid may alternatively be formed from a round or oval profiled wire, a ribbon is preferred because of the overall lower profile attainable for an enhanced amount of kink resistance. The ribbon is preferably less than 1.5 mil in thickness, more preferably 0.7 mils to 1.5 mils, most preferably about 1 mil. The width desirably is 2.5 mils to 7.5 mils in width, more preferably about 5 mils.

By “braid” here, we mean that the braid components are woven radially in and out as they progress axially down the braid structure. This is to contrast with the use of the term “braid” with co-woven coils merely laid one on top of the other in differing “handed-ness.”.

Returning to the discussion of the anti-kinking member (150), the antikinking member (150) may also suitably be a wire of suitable cross-section, e.g., round or oval or square. It need not be wound from one end of the catheter to the other, over the junctions between regions of different diameter, but it is desirable to do so. Anti-kinking members (150) may simply be multiple coils co-wound at the same time. Other variations include braids and multiple coils wound in opposite directions. The single layer ribbon coil is highly desirable because of the ease of assembly in placing the coil upon a catheter subassembly, particularly when the catheter subassembly has a variety of diameters. The other advantages include a high measure of kink-resistance even with an extremely low profile.

The joint between the stiff proximal section (136) and the significantly more flexible distal section (132), as shown, incorporates an exceptional amount of strain relief without being bulky. In particular, the joint involves the stiffest inner member (142), perhaps the transition section (144) and the soft flexible covering (134). Central to the strain-resisting feature is the use of a corkscrew shaped section of material (152) that extends over the joint. In the event a coil or other similar strain relief device is employed, the added high strength corkscrew (152) is desirably placed between the turns of the anti-kinking device (150). Of course, the outer layers of shrink-wrap tubing (134 and 148) are also desirable in providing strength to this joint.

Distally on the variation in FIG. 2 may be found the balloon (138) access passageways (154) and tip seal (140). The balloon (138) is desirably of a highly compliant polymeric material, preferably an elastomeric stretchable material such as silicone rubber, latex rubber, polyvinylchloride (PVC), chloroprene, or isoprene. Radiopaque markers both (156) proximal of the balloon and (158) distal of the balloon (138) are also shown. In this variation, each of these markers (156, 158) is shown to be coils of a radiopaque material such as platinum or alloys of platinum/iridium and other suitable materials.

As is apparent from the FIG. 2 drawing, when seal (140) is closed, e.g., by introduction of a closely fitting guidewire, introduction of fluid through the open lumen of catheter (130) will cause fluid to flow through orifices (154) and expand balloon (138).

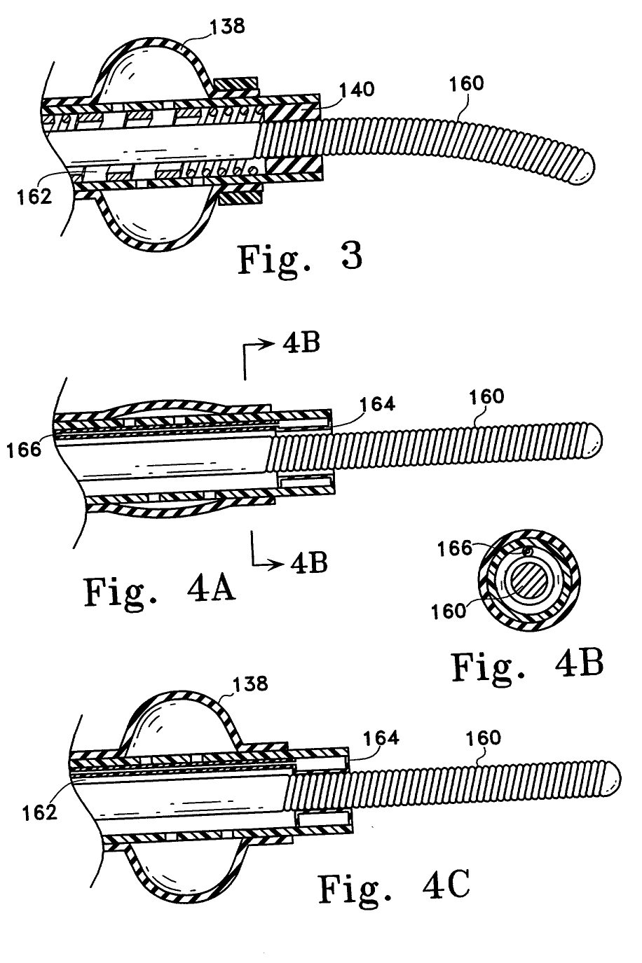

FIG. 3 shows guidewire (160) in contact with and closing seal (140) thereby causing balloon (138) to expand upon introduction of fluid into lumen (162).

FIGS. 4A, 4B, and 4C show a variation of the inventive catheter in which the seal (164) is expandable. A separate inflation lumen (166) is also shown. The benefits of this variation are many. Specifically, the guidewire (160) is free to move both longitudinally and without significant friction from the seal during placement of the catheter using that guidewire (160). Once the seal (164) is inflated as is shown in FIG. 4C, the lumen (162) is tightly and controllably closed for use in inflating balloon (138). Depending upon the design, annular inflatable seal (164) may “freeze” the guidewire (160) in place allowing the catheter—guidewire assembly to move easily as a single unit.

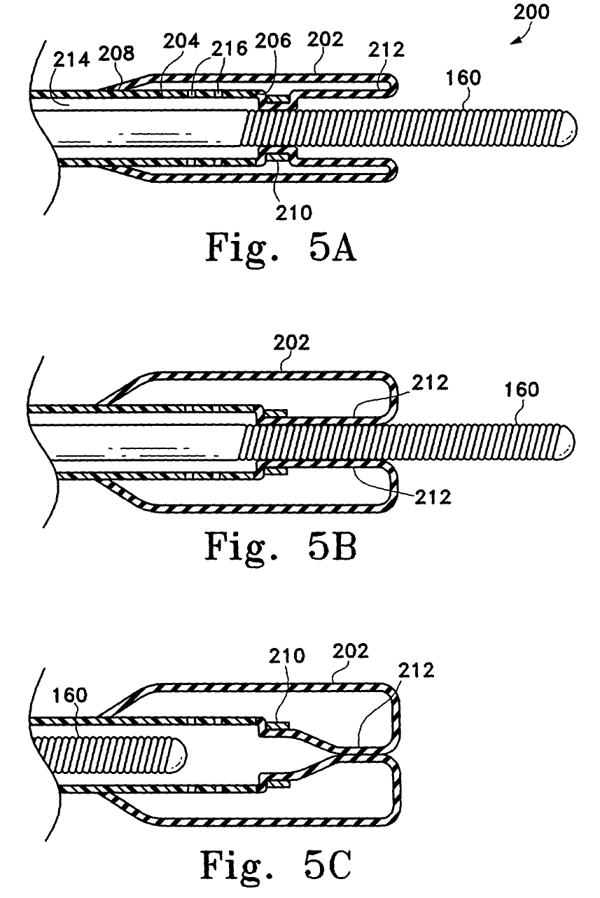

FIGS. 5A through 5C show another variation of the inventive catheter (200). The distal tip including balloon (202) is also shown in FIGS. 5A 5C. In this variation, the balloon material is attached at the end of a stiffer tubular member (204) at, e.g., joint (206). Although the joint is shown here to be a butt-joint, the joint may be other joint structures as is appropriate for this kind of balloon assembly. This variation also uses an everted balloon (202) that folds back and is attached to the distal tubular member (204) at joint area (208). Finally, an auxiliary seal region (210) is implemented in this variation. The primary seal is the distal region of the balloon (212) as will be explained in more detail with regard to FIG. 5C. Inflation fluid flows from the annular space (214) through the orifices (216) into the chamber of the balloon (202).

FIG. 5A shows the balloon in a deflated condition prior to the time inflation fluid is introduced through orifices (216). Auxiliary seal (210), however closes the annular space (214) to substantial flow of inflation fluid other than into the balloon. FIG. 5B shows the balloon (202) in an inflated condition. It should be noted that the distal regions of the balloon (212) act as the primary seal and are closed against guidewire (160).

FIG. 5C shows the inflated balloon (202) with the guidewire (160) withdrawn from contact with the balloon (212) and the auxiliary seal region (210). Auxiliary seal (210) may be either compliant and in the form of an elastomeric ring much like a small rubber band or may be a properly sized rigid ring of a metal or other suitable material. In any event, primary seal region (212) remains closed and both the inflation of and deflation of the balloon are then controllable only by withdrawal of or introduction of fluid from annular space (214).

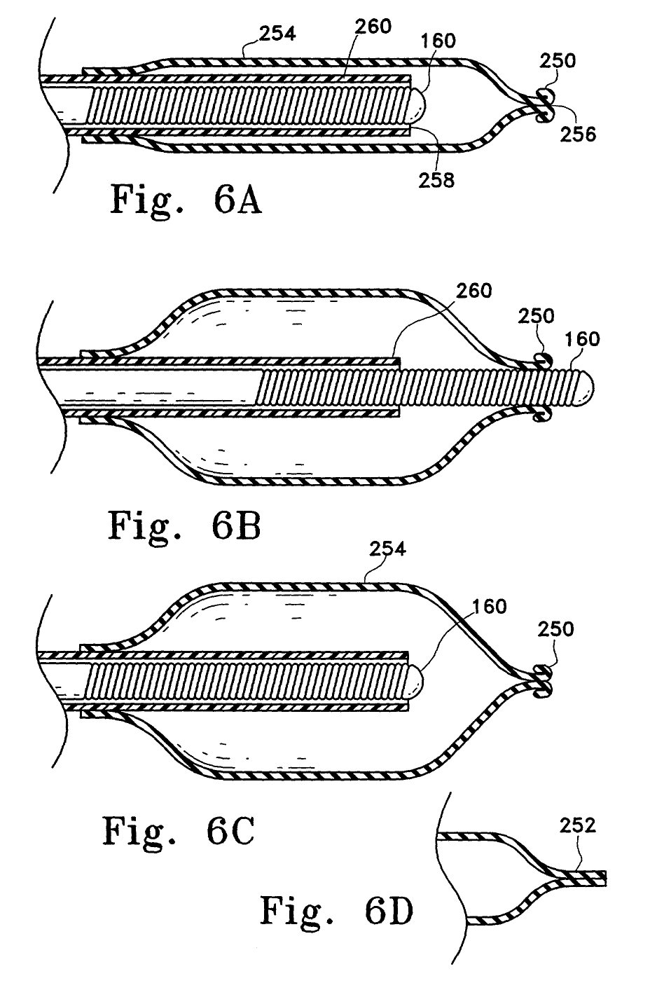

FIGS. 6A and 6B show two versions of the inventive catheter in which the seal regions are located distally of the balloon and are normally closed. These variations also permit sealing of distal seal (250 in FIGS. 6A, 6B, 6C and 252 in FIG. 6D) also seal upon the included guidewire (160).

FIG. 6A shows a deflated balloon (254) having a distal seal region (250). The seal region (250) is everted in that it is folded back upon itself but retains an orifice (256) through which guidewire (160) may pass. In this variation, inflation fluid flows out of distal tip (258) of catheter body (260). Seal region (250) is self sealing and upon introduction of fluid through the catheter, will inflate whether the guidewire (160) is present in seal (250) or not. Similarly, as may be seen in FIG. 6C, withdrawal of the guidewire from seal region (250) will not cause deflation of balloon (254). Production of small everted seals such as that shown in (250), is reasonably simple in that the open end may be simply rolled back and, e.g., glued to itself to form one or more layers of balloon material in that region. FIG. 6D shows an alternative seal region (252) that involves only a single layer of material.

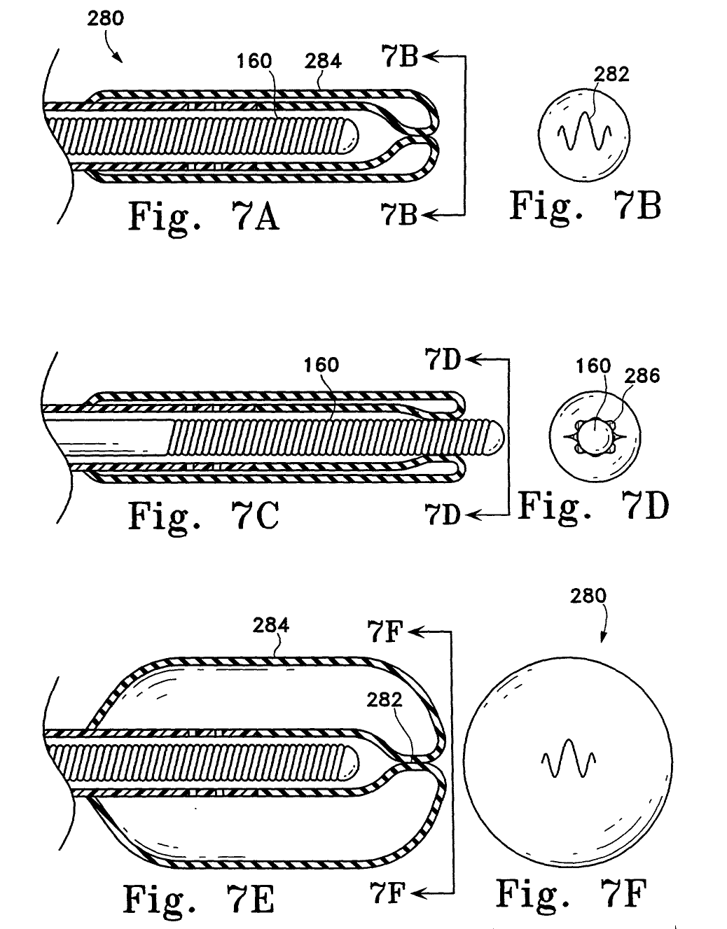

FIGS. 7A through 7F show still another variation of the inventive catheter (280). In this variation, guidewire (160) is used in cooperation with the shape of the seal region or mating surfaces in such a way that introduction of guidewire (160) into the seal surface will permit the balloon to be deflated.

FIG. 7A shows deflated balloon (284) prior to introduction of inflation fluid to the balloon and, the presence of guidewire (160). FIG. 7B shows an end view of catheter with its convoluted or sinusoidal seal mating surface (282).

In FIG. 7C, guidewire (160) has been extended through seal mating surface (282) creating a number of openings (286) along the outer surface of guidewire (160). Fluid flows through these openings for deflation of the balloon. The protuberances of seal region (282) push against guidewire (160) to enhance the opening flow spaces (286). It is desirable that the material in the seal region (282) have a bit higher stiffness than the material of the surrounding balloon to allow for creation of the flow areas (286).

FIG. 7E shows a partial side view of the inflated balloon (284) as sealed by seal region (282).

FIG. 7F shows an end view of the inflated device (280) as otherwise shown in FIG. 7E.

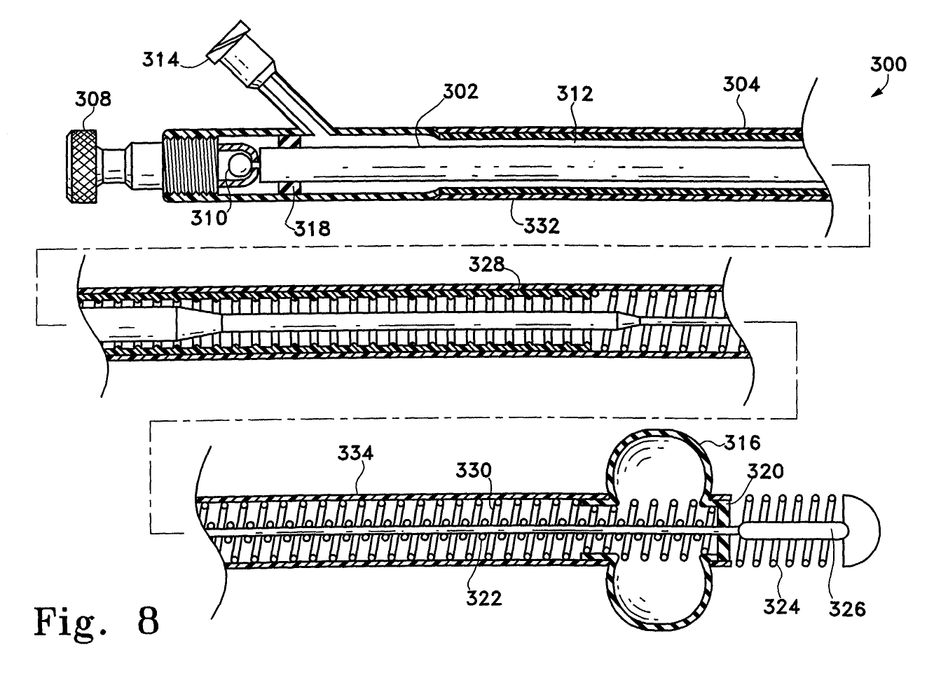

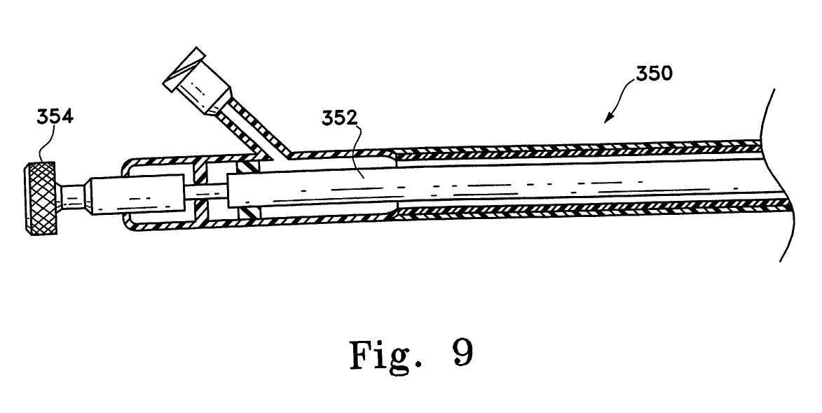

FIGS. 8 and 9 show an additional variation of the inventive catheter (300). This variation includes a guide wire or core wire (302) that preferably is ground in such a way so to allow its use in conjunction with the surrounding catheter body (304) as a guide wire. More particularly, the core wire (302) is not removable from catheter body (304) during normal usage. The function of the core wire (302), in addition to its utility as a way for the device to be used in a “guiding” fashion, is that the core wire may be used to provide a measure of additional stiffness by an axial “pulling” on core wire (302). By controlling the overall flexibility of the device by twisting knob (308), the flexibility may be incrementally and continuously varied. It is highly desirable that the adjustment component (308) be configured in such a way that it does not transmit torque to core wire (302). The ball and socket joint at (310) is one way to prevent substantial torque from being transmitted to core wire (302). The annular region (312) for passage of inflation fluid from fluid import fitting (314) to balloon (316) is isolated by seal (318) at proximal end and seal (320) at the distal end. Other desirable features of this particular variation include the use of radio-opaque marker coils (322) coincident with and proximal of balloon (316) and a shapeable radio-opaque coil (324) distal of balloon (316). Desirable, but not required, is the flat region (326) of core wire (302) to allow an initial manual bending of the tip of the device for usage as a guide wire.

The other functional structure of this variation of the guide wire may generally be as shown above with respect to the other catheter bodies. The use of anti-kinking or stiffener members (328) (flat wound ribbon coil) and a wire coil (330) may be as discussed above. The polymeric materials forming the desirably stiffer proximal end (332) and the more flexible materials making up distal end (334) may be also be as discussed above. Similarly, the inflatable balloon (316) may be produced from the materials discussed above and those otherwise used in compliant balloons in this art.

It is preferable that the balloons used in this device be compliant, that is, elastic in that typically they are used in the vasculature of the brain and high pressure is not always desired. The diameter of the balloons, once inflated, may also be controlled with such a balloon design. However, fixed diameter balloons are certainly within the scope of this invention, just not preferred.

FIG. 9 shows variation (350) of the device shown in FIG. 8, with the exception that the stiffening of the guide wire by axial or longitudinal movement of that core wire (352) is more pronounced and not as finely adjustable as is the variation in FIG. 8. Simply pulling on knob (354) will produce stiffening of the distal end of the catheter (350). Otherwise, the device is as otherwise described above.

One of the uses of this device will be in the placement of vasoocclusive devices and materials in aneurysms. Vasoocclusive coils and the like (such as the Guglielmi Detachable Coil or “GDC”) are well-known and widely used. However, it is also highly desirable to include other occluding materials such as cyanoacrylates and partially hydrolyzed polyvinyl acetate and the like in such aneurysms, particularly when the aneurysm is a wide-necked one. However, it is not always easy to maintain continuous or sticky or reactive media such as cyanoacrylate glues in a wide-mouth aneurysm. The inventive catheter is ideal for maintaining these materials in the aneurysm until they are effective in occluding the aneurysm, but the balloon and its environs must be inactive with regard to the occluding material.

This invention has been described in reference to various illustrative embodiments. However, this description is not intended to be construed in a limiting sense. Various modifications and combinations of the illustrations, as well as other embodiments of the invention, will be apparent to those persons skilled in the art upon reference to the description. It is therefore intended to be appended to claims encompassing any such modifications or embodiments.

We claim as our invention:

1. A low profile balloon catheter for use with a removable guide wire comprising:
A catheter body having a distal end, a proximal end, and a passageway for inflation of a balloon at the distal end of the inflation lumen,
said balloon located near said distal end, and
a movable seal adapted to cooperatively seal said passageway and to inflate said balloon upon introduction of fluid into said passageway.

2. The catheter of claim 1 wherein the movable seal is inflatable by a fluid supply lumen independent of said inflation passageway.

3. The catheter of claim 2 where the movable seal is situated within said passageway and upon inflation of said seal closes against said removable guide wire.

4. The catheter of claim 1 wherein the movable seal is both self-closing and sealable against the removable guide wire when said guide wire passes through said movable seal.

5. The catheter of claim 4 where said seal is distal of said balloon.

6. The catheter of claim 5 where said seal is a continuous extension of said balloon.

7. The catheter of claim 6 is an everted extension of said balloon.

8. The catheter of claim 1 further comprising an auxiliary seal for initially sealing said passageway against said guide wire and where the balloon includes a distal end forming said seal, both against said guide wire and self-closing against itself upon introduction of a fluid into said balloon.

9. The catheter of claim 1 where said seal is adapted to close said passageway and inflate said balloon when said guide wire does not pass through said seal and when fluid is introduced into said passageway and said seal is adapted to allow passage of fluid through said seal and deflation of said balloon when said guide wire passes through the seal.

10. The catheter of claim 9 where the seal has a mating surface adapted to cooperate with said guide wire to provide openings in said seal adjacent said guide wire.

11. The catheter of claim 10 wherein said mating surface is sinusoidal.

12. The catheter of claim 1 wherein the balloon is compliant.

13. The catheter of claim 1 wherein the catheter body has a catheter shaft with a proximal section different that said distal section.

14. The catheter of claim 13 where the proximal section has a larger diameter than the distal section.

15. The balloon catheter of claim 1 wherein said catheter is of a flexibility, length, and diameter appropriate for a neurovascular microcatheter.

16. The balloon catheter of claim 1 wherein said catheter is of a flexibility, length, and diameter appropriate for a guide catheter.

17. A low profile balloon catheter comprising:
a catheter body having a distal end, a proximal end, a flexible distal section, and a passageway for inflation of a balloon at a distal end of the inflatable lumen,
an inflatable balloon located near said distal end,
a non-removable guide wire,
a seal situated in said passageway adapted to seal against said guide wire for inflation of said balloon upon introduction of fluid into said passageway, and said guide wire extending distally of that seal.

18. The catheter of claim 17 wherein the guide wire is adapted to provide axial stiffness to said flexible distal section.

19. The catheter of claim 18 wherein the stiffness of the guide wire is continuously and incrementally variable.

20. The catheter of claim 17 where the guide wire has a variable diameter.

21. The catheter of claim 17 where the proximal portion of said catheter is a hypotube.

22. The catheter of claim 17 wherein said catheter is of a flexibility, length, and diameter appropriate for a neurovascular microcatheter.

23. The catheter of claim 17 wherein said catheter is of a flexibility, length, and diameter appropriate for a guide catheter.

24. A strain relief joint between a first comparatively stiff section adjacent, a second comparatively flexible section comprising said first stiff section, said flexible second section joined to said first section at a joint, and a corkscrew-shaped component wound over said joint and adherent both to said first and to said second sections.

25. The strain relief joint of claim 24 wherein said first and second sections are tubing members.

26. The joint of claim 25 where the first and second sections are polymeric.

27. The joint of claim 26 wherein said joint is at least partially wrapped by a metallic ribbon.

28. The joint of claim 27 wherein the metallic ribbon comprises stainless steel alloy.

29. The joint of claim 28 where the first stiff section has a diameter different than the second flexible section.

30. The joint of claim 29 where the metallic ribbon and the corkscrew-shaped component are adherent to the tubing sections via a molten and solidified polymer.

31. The joint of claim 29 further comprising one or more shrink-wrapped polymeric coverings.
